(19) United States
(12) Patent Application Publication
(10) Pub. No.: US 2006/0025326 A1
(43) Pub. Date: Feb. 2, 2006

(54) Method for treating carrier particles and its use

(75) Inventors: Tapio Lankinen, Turku (FI); Heikki Salonen, Turku (FI)

Correspondence Address: ROTHWELL, FIGG, ERNST & MANBECK, P.C., 1425 K STREET, N.W., SUITE 800, WASHINGTON, DC, 20005, US

(73) Assignee: LAB Pharma Oy, Turku (FI)

(21) Appl. No.: 10/509,256

(22) PCT Filed: Mar. 28, 2003
(86) PCT No.: PCT/FI03/00241

§ 371 Date: Jun. 14, 2005

(30) Foreign Application Priority Data
Mar. 28, 2002 (FI) 20020607

(51) Int. Cl. A61K 38/00

(52) U.S. Cl. 514/2

(57) ABSTRACT

A method for treating a particulate carrier for an inhalation powder improving the stability and flowing properties of the carrier. The carrier is abraded suspended in a liquid medium, in which the carrier is essentially insoluble, the liquid medium is evaporated and the carrier recovered.

This application is a National Phase of International Application Serial No. PCT/FI03/00241, filed Mar. 28, 2003.

### FIELD OF THE INVENTION

The present invention relates to a method for treating a particulate carrier for an inhalation powder improving stability and flow properties of the carrier. The invention further concerns the carrier and a pharmaceutical preparation for inhalation purposes containing said carrier.

### BACKGROUND OF THE INVENTION

Micrometer-millimetre size particulate materials, organic or inorganic, are normally not spherical nor rounded but rather edged or rough surfaced after crystallisation or other manufacturing methods.

There are some known methods to modify the shape of carriers. In U.S. Pat. No. 6,153,224 classified lactose particles were gently milled in a ball-mill in order to remove the asperities on the surface as small grains, which will be reattached on the high-energy sites of the carrier. When a small amount of a ternary agent (L-leucine) was milled with lactose, the grains of the agent adhered to lactose. As a result, such a modified carrier showed decreased adhesion to drug particles and hence, better small particle fraction in laboratory tests was achieved.

Iida et al. (Chem. Pharm. Bull. 49(10) 1326-1330 (2001) Vol. 49, No. 10) removed protuberances from the surface of lactose carrier by controlled dissolution. The resulting particles were rounder and without sharp edges compared to untreated lactose. Drug mixtures made of such lactose showed improved flow properties and better fine particle fraction, when packed in capsules and used in a powder inhaler.

No methods concerning carrier polishing with a high energy disperser or a corresponding mixing device based on vigorous mixing of a suspension has, however, been described.

The patent application WO 02/00197 A1 Staniforth et al. discloses a method for making microfine composite particles. This is done preferably by wet-milling the components in a ball mill. It is also mentioned that a high-energy liquid homogenizer can be used for the purpose. In this connection, polishing of larger carriers in not mentioned.

Microencapsulation of carriers by spray drying and some other methods for coating carriers have been reported, but these include no polishing or abrasion of the carrier surface.

### SUMMARY OF THE INVENTION

To improve the separation of carrier and active particle the invention suggests that carrier is abraded suspended in a liquid medium into which the carrier is essentially insoluble, the liquid medium is removed and the carrier recovered. The so abraded or smoothened carrier particles have been found to more efficiently deliberate the active particles adhered to the carrier. Also the physical stability of the treated carrier is enhanced. The flow properties of the treated, filtered and dried carrier were clearly improved.

The abrasion is preferably conducted with a mixer device such as a high performance disperser using an effect below that required for crushing the carrier particles thus avoiding breaking up the particles to be treated. Preferably the abraded carrier is at least partly covered with fine particles.

The invention also concerns a carrier for an inhalation powder, which carrier is stable and possesses good flow properties, characterized in that the carrier is abraded suspended in a liquid medium, in which said carrier is essentially insoluble.

A further feature of the invention is a preparation for inhalation purposes comprising an active agent, a carrier and optional excipients used in inhalation preparations. The carrier in this preparation is at least partly abraded suspended in a liquid medium, in which the carrier in essentially insoluble. An especially advantageous preparation contains in addition to the abraded carrier also a micronised carrier. Such preparation has even a more prolonged shelf-life than a preparation manufactured of drug and polished carrier alone.

### DETAILED DESCRIPTION OF THE INVENTION

The particle characteristics of the raw materials strongly affect the final quality of the pharmaceutical product and therefore very strict requirements are applied on these properties in the pharmaceutical industry. A special case of such material science in pharmaceutical industry is the formulation for a powder inhaler. The overall performance of the powder inhaler is highly dependent on the characteristics of the powder components, e.g. particle size distribution, crystal morphology, shape and surface roughness of the particles and interparticle attraction forces, including static charges. An important aspect which must be taken into account with handling and processing of inhalation powders is the prevention of microbial contamination.

Delivery of drugs into the lungs is common in asthmatics and is usually done with a metered dose inhaler (MDI; pressurised aerosol) or a powder inhaler. Irrespective the dosing device, the delivered particles must be no larger than 5 micrometers in respect of the tested MMAD (mass median aerodynamic diameter) in order to be able to deposit in the deep lung. It is expected that the use of powder inhalers will strongly increase and systemic delivery of new drugs, including large molecules, will be a marked target for powder formulation technology.

The classical powder formulation consists of an air-jet milled micronsize drug and a carrier sugar, commonly lactose monohydrate. The mean particle size of the carrier is mostly between 50 and 100 microns and the particle size distribution is broad resembling a Gauss' curve. The maximum size is about 300 microns, larger may cause irritation in upper airways. The form of commercial lactose is typically an elongated triangle, called “tomahawks”. Glucose can be used as carrier and the particles do not differ markedly from lactose in respect to the particle size distribution or particle form. Mannitol, sucrose and trehalose are under investigations for carrier sugars. Classified samples are offered for test purposes by some sugar manufacturers and such carrier materials will be available for innovators in manufacturing scale.

The drug content in a powder formulation is typically less than 10 w-%. It is calculated that with a drug concentration of 5-10% the carrier particles are covered by a uniform layer of drug particles. Higher concentrations may cause segregation and will demolish the flow properties of the formulation. Excellent flow properties are extremely important for multiple dose powder inhalers (MDPIs), where each dose must be accurately metered by manoeuvres done by the patient. Capsule filling of unit dose devices or filling of the blisters for blister-based devices can be done with formulations having poorer flow properties.

There are some main factors which regulate the performance of the formulation in use:

Force of Attraction Between Drug and Carrier Particles

Micronsize particles tend to adhere very strongly to each other and carrier particles. During inhalation the drug particles should be liberated again in order to be able to go into the deep lung. This disintegration takes place due to turbulence, shear and centrifugal forces. If the carrier particles are rough and edged, drug particles may be hidden in the high-energy corners and cavities and they are resistant to shear and turbulent flow. The degree of loose drug particles can be easily metered with a cascade impactor, where the fine particle fraction (FPF %) and mass median aerodynamic diameter (MMAD) of the delivered drug particles can be calculated.

Van der Waals forces are important in respect to the adherence when small distances are concerned. If two particles have a direct contact with large contact area, the attraction is high and they are difficult to separate. If there is a primary layer of very small particles on the carrier surface, the secondary drug particles tend to adhere relatively loosely on the carrier surface due to diminished Van der Waals forces. It means that one can increase the FPF % if very small particles are mixed to the formulation, preferably before the drug particles. Ideally, the material is the same which is used as the coarser carrier. In literature, the particle size of added small particle carrier has been about the same as the particle size of micronised drug.

If the surface of any of the formulation components is modified, the performance of the product will change due to altered particle-particle interactions. This refers also to particle mean size and particle size distribution of the components.

Physical Stability of the Components

The components should be physically stable or in their thermodynamically lowest energy level. If not so, the component will change its physical state more or less slowly, accelerated by increase in temperature and humidity. The change is seen as altered performance and is a common reason for impaired shelf-life of the product. Air-jet milling of the drug creates easily amorphous material on the surface of drug particles. Vigorous dry mixing may do the same to all components. The formation of amorphous material is highly drug specific. Some drugs may transform into a totally amorphous state whereas some do not change at all. It is commonly believed that the amorphous content in the micronised drug is mainly responsive for the impaired physical stability of inhalation powders. The role of the carrier has remained more unclear in this respect.

Factors Affecting Dose Accuracy

Concerning MDPIs, the accuracy of the dose metering mechanism of the device is decisive. In most cases the dose is metered to a dose slot or slots to be transferred to inhalation air stream. This volumetric dose metering may work accurately only, if the formulation shows proper and unaltered flow properties through the shelf-life. If the formulation is not physically stable, changes in the morphology may cause agglomeration of the powder, followed by impaired flow properties and dose accuracy. If there are too many micronsize particles (more than 10 w-%), in the formulation, the flow properties may initially be impaired and the formulation is even more sensitive to further disturbances, e.g. to unstability of some component. In respect to flow properties, the sensitivity of the dose metering system may vary between different MDPIs.

Ideally, the best dose accuracy and longest shelf life is obtained if the formulation is physically stable, it is protected against moisture with a desiccant and the flow properties remain unchanged in actual use circumstances.

It was discovered that edged and rough carrier sugars could be rounded and polished by treating a carrier suspension some hours with a high performance disperser. By introducing the suspension through a classifying filter pack during the procedure a certain range of particle size of polished sugar could be obtained. Drug formulation made of polished carriers showed enhanced performance and characteristics, especially in physical stability, when used in a MDPI.

Description of the Test Methods.

The first experiments were done by treating the n-hexane (Mallinckrodt Baker B V, the Netherlands) suspension of Pharmatose® 325 M lactose monohydrate (DMV, The Netherlands), mean particle size 60 microns, for some hours with an Ultra-Turrax® high performance disperser IKA T 25 Basic (20.000 rpm) (IKA GMBH & Co KG) in a decanter. The batch size was some tens of grams. It was found that up to 30% of the initial amount of lactose was abraded to micronsize particles, which could be filtered away. In the next step the disperser was provided with a flow-chamber, suspension vessel and with an ice-bath cooled recirculation line. The product was obtained by filtering the treated suspension through a 40 micron filter, followed by vacuum drying.

The pilot-scale polishing equipment is based on IKA SD 41 Super-Dispax® high performance disperser (IKA GMBH & Co KG), equipped with a flow-chamber for circulation of the feed suspension. An on-line filter pack was used to separate smaller than 40 micron particles as waste and return the larger particles to the flow-chamber. The principle of the filter is explained further in U.S. Pat. No. 6,027,656. The filter principle enables to recirculate or vaste more than one main range of particle size, if more than two filter planes are used.

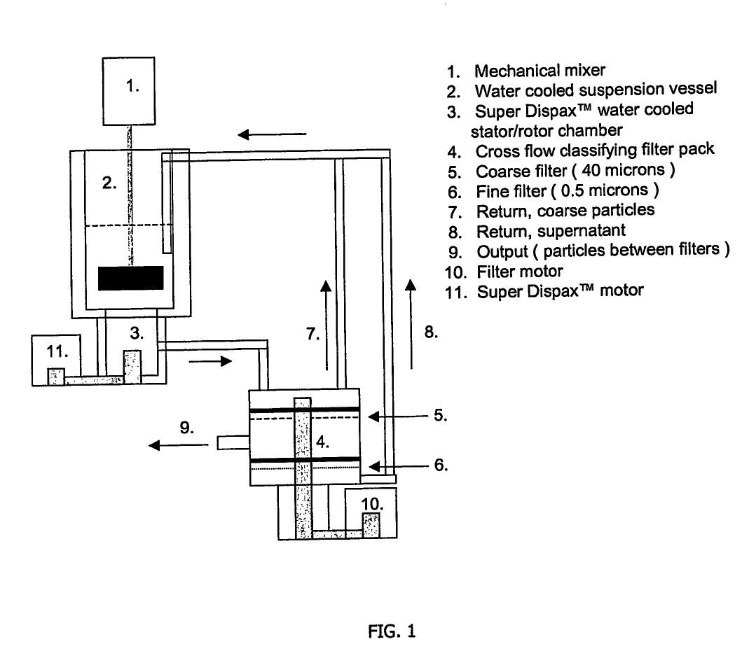

The system is illustrated schematically in FIG. 1 presenting a pilot scale polishing equipment.

A water cooled suspension vessel 2 is equipped with a mechanical mixer 1 and below a Super Dispax® water cooled stator/rotor chamber 3 equipped with a motor 11. The suspension obtained in vessel 2 is fed to the chamber 3 (flow-chamber) for the rotor/stator treatment and then to the filter device 4 with a motor 10 and two filters, the first one being a coarse filter 5 (pore size 40 μm) and the second one is a fine filter (pore size 0.5 μm). An abraded screened product is obtained from filter output 9. Coarse fraction 7 and the very fine fraction 8 are returned to vessel 2.

Three different liquids were tested as lactose suspension: n-hexane (Mallinckrodt Baker BV, the Netherlands), 2-propanol (Mallinckrodt Baker BV, the Netherlands) and a mixture of non-flammable perfluoroethers [Galden® (Ausimont, Italy)]. Even if the densities of the liquids were very different (n-hexane=0.7 g/cm3, 2-propanol 0.8 g/cm3, Galden® 1.6 g/cm3), no difference in the polishing efficacy was found.

A validation program of the system was carried out with Pharmatose® 110 M lactose monohydrate (DMV, the Netherlands) and 2-propanol as the suspending liquid. The studied parameters were lactose:2-propanol ratio, roundness (shape factor), RPM of the rotor and the distance of stator/rotor. The coarse filter was 40 μm and the fine filter 0.5 μm. The time of the treatment was 3 hours.

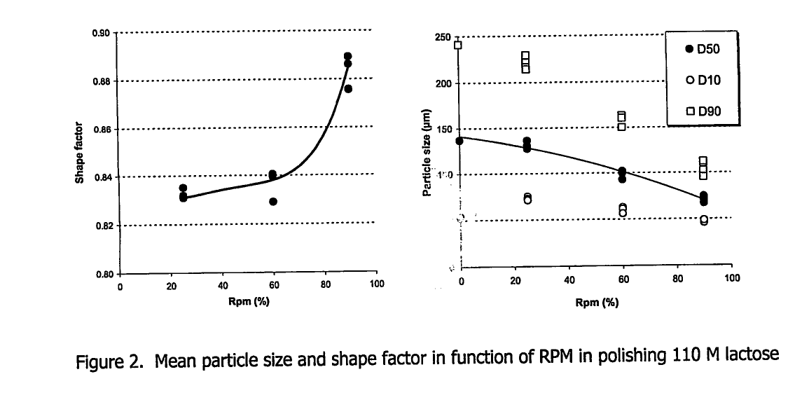

The results revealed that only RPM of the rotor is of practical importance. In practice, 100 g carrier/1 litre produces a good suspension. Distance of stator/rotor should be at least twice the diameter of the largest particles. If not, the particles are crushed and not polished. Results on the effect of RPM on particle size (10%, 50% and 90% with smaller size) and roundness are seen in FIG. 2.

The desired particle size may be obtained by choosing the appropriate rotor/stator distance and/or rotation speed of the mixer. Said distance is material specific, for greater particles a distance of 0.5 mm may be sufficient, whereas the distance may be reduced if smaller sized particles are treated. The higher the rotation speed is the smaller is the resulting average particle size. Finally, the treatment time is decisive.

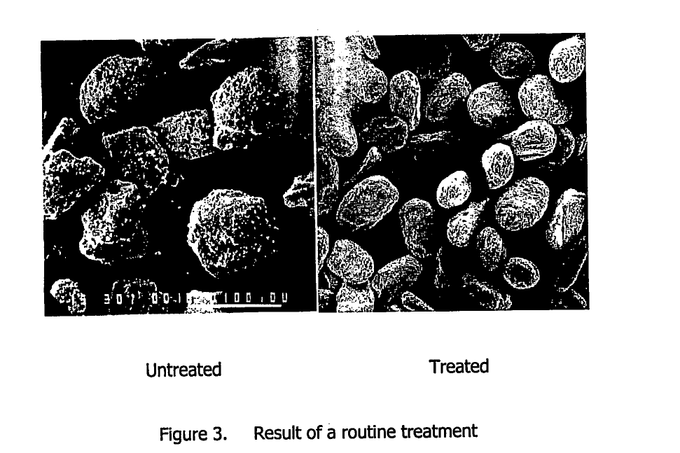

One should use at least 60% of the maximum rotor speed (13.000 RPM) and at least 80%, if maximum roundness is wanted. However, if also maximum polishing is wanted, one should use first RPM of at least 80% and after that some hours with 60% or less, when the final polishing is done gently and gives smoother surface. The result of a routine treatment of 110 M lactose in 2-propanol (3 h, 80% RPM) is seen in FIG. 3.

Another example is explained, when 110 M glucose anhydrous (Kirsch Pharma GmbH, Germany) was treated in Galden® 100 (Ausimont, Italy): Ultra Turrax® basic provided with a flow-chamber and ice-bath cooled recirculation was used as a disperser. Treatment time was 1.5 h, disperser speed was 22.000 RPM and the amount of glucose anhydrous was 150 g. 1500 ml of GALDEN® 100 was used as medium.

After the treatment the suspension was filtrated through a 40 micron filter and was washed several times with n-hexane in order to remove the residual small particles. Then the filtered mass was dried in vacuum. The dry product was sieved through a 150 micron sieve.

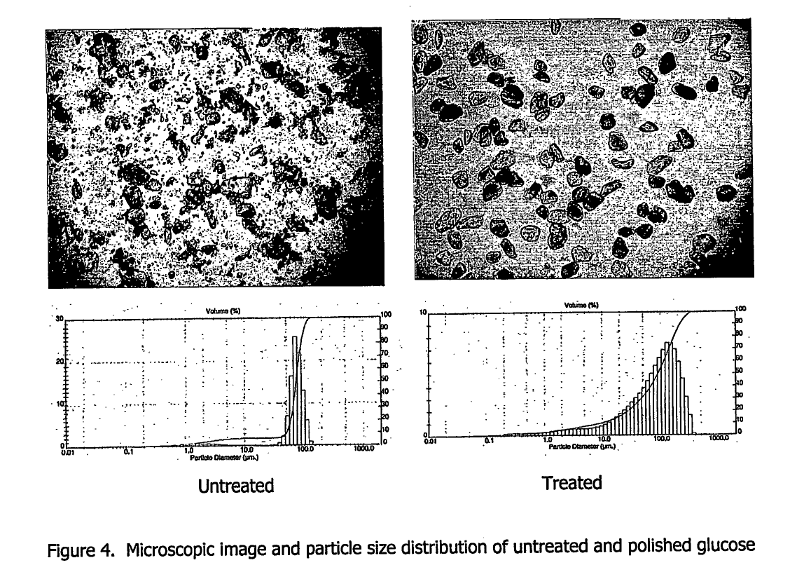

Microscope photos and particle size distributions of the starting material and the final product are seen in FIG. 4.

According to microcalorimetric studies, untreated lactose contained detectable amounts of unstable (amorphous) material. When the study was repeated with polished lactose of the same manufacturing batch, no signs of amorphous material were found. It is obvious that the amorphous matter was located at the surface of lactose and was removed by polishing. Lack of amorphous substance on the surface of polished carrier particles is most obviously the reason for enhanced stability of the final formulations. The improvement in stability was surprisingly clear and indicates the importance of the carrier in addition to the micronised drug in respect to physical stability of the formulation.

Test Results with Formulations Containing Polished Carrier

The tests were carried out as follows: The formulations were manufactured by the wet-mixing method explained in Finnish patent No. 105.078 and contained the active drug and Pharmatose® 110 M lactose monohydrate carrier untreated or polished. The mean particle size of the polished carrier was about 60 microns and there were no significant amounts of smaller than 40 micron particles. The formulations were stored one week at 25° C./33% RH and were then packed in two TAIFUN® MDPIs for testing the initial performance of the products. Two polycarbonate tubes were filled with the same powder and placed immediately to stress circumstances of 45° C./75% RH for one month. The tubes are permeable to moisture and do not shelter the formulation. Then two TAIFUN® MDPIs were filled with the formulation and tested.

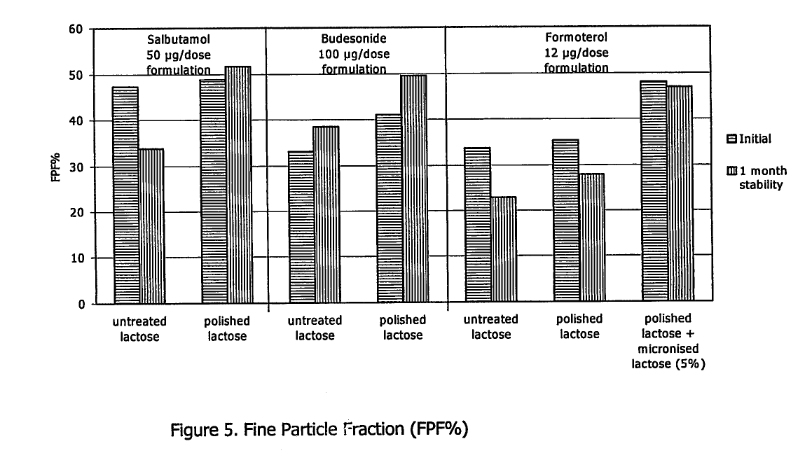

The tests were done using an Andersen cascade impactor at constant ambient circumstances 25° C./60% RH. The main parameter was fine particle fraction, which is the percentage of smaller than 5.8 μm drug particles of the total delivered dose. Each result is the mean of two tests. The dose strengths of the formulations were: salbutamol 50 μg/dose, formoterol 12 μg/dose and budesonide 100 μg/dose. The results are seen in columns shown in FIG. 5 and explained below.

Salbutamol Formulations

When untreated lactose was used, the initial FPF % is rather good (over 45%), but considerable reduction to less than 35% takes place during storage. For polished lactose, the initial value is almost 50% and improves to over 50% during storage.

Budesonide Formulations

Initially the FPF value for untreated lactose is not good but improves somewhat during storage. For polished lactose the behaviour is very similar, but the FPF values are clearly better (average 35 contra 45%). It is obvious that lipophilic budesonide stands stress better than hydrophilic salbutamol with some amorphous material in it. Also, no amorphous fraction was detected in budesonide in a microcalorimetric study. However, the effect of polished carrier is clear.

Formoterol

With untreated lactose the initial value is acceptable but falls down during storage. With polished lactose the result is clearly better, even if a small depletion in the FPF % is seen during stress. The best results were obtained, when a small amount of micronised carrier (5 w-% of total) was mixed in the formulation. Now the FPF % was excellent and did not change when stressed.

It is commonly known that formoterol is difficult to formulate as inhalation powder; the FPF % is low and the physical stability is questionable. In this work it was detected that formoterol contains percents of amorphous material, which is difficult to recrystallise beforehand. By increasing the temperature for on hour to 60° C. as hexane suspension (as described in patent FI 105.078) most, but not all of the unstable material, recrystallised. This find may explain the difficulties.

Polishing Optimation

The polishing optimization parameters for lactose were determined with a device described earlier in the specification. The parameters involved were rotation speed of the rotor, the distance between rotor and stator, amount of suspension medium (ethanol). The polishing time was 3 hours and the amount of lactose 400 g.

The parameters used are listed in the table below:

POLISHING OPTIMIZATION
PARAMETRES

| | rpm | stator/rotor | amount of |
|---|---|---|---|
| Test, | (max 13000), | distance | suspension medium |
| 1. | 60% | 0.5 mm | 4000 ml |
| 2. | 25% | 0.3 mm | 6000 ml |
| 3. | 60% | 0.5 mm | 4000 ml |
| 4. | 60% | 0.5 mm | 4000 ml |
| 5. | 25% | 1.0 mm | 2000 ml |
| 6. | 90% | 0.3 mm | 2000 ml |
| 7. | 90% | 0.3 mm | 6000 ml |
| 8. | 25% | 1.0 mm | 6000 ml |
| 9. | 25% | 0.3 mm | 2000 ml |
| 10. | 90% | 1.0 mm | 2000 ml |
| 11. | 90% | 1.0 mm | 6000 ml |

Polishing time: 3 h; Amount of lactose: 400 g

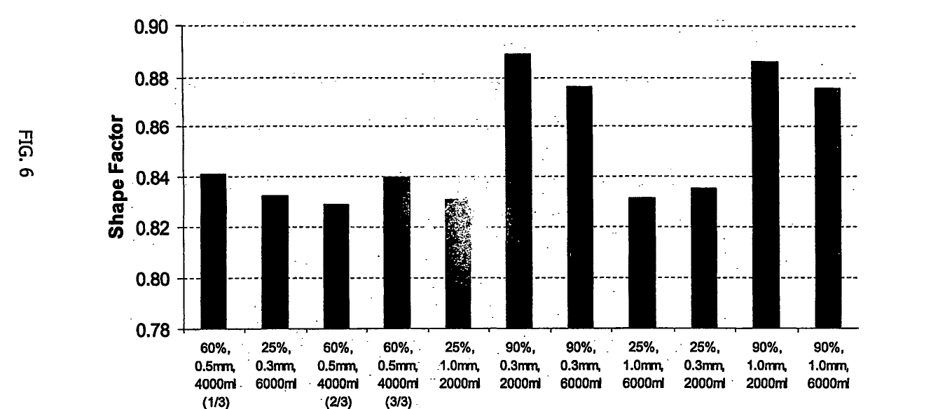

The results obtained presented in FIG. 6 reveal the rotation speed is the most important factor when high shape factors are desired.

Increase of micronsize material to the carrier is a well-known method to increase the FPF %. In EP 663.815 this method was utilised to increase FPF % when the formulation was used in a capsule based device. However, the flow properties of such a formulation are too impaired for use in multi-dose powder inhalers. Tests with Taifun MDPI showed that increase of micronised material to a formulation based on commercial lactose grades for inhalation worsens the flow properties, followed by diminished dose and worse dose accuracy.

For acceptable dose accuracy, the total amount of micronised substance should not exceed 15 w-% of total. Polished carriers offer a new opportunity to benefit increase of micronised carrier to enhance both the FPF % and physical stability. Accordingly, the flow properties stay acceptable to be used in MDPIs, because the tested dose accuracy of the formulation with added micronsize carrier was excellent with an RSD % value of 7.

The method accepts different carrier materials, eg. glucose and mannitol were tested successfully in this work. Concerning the suspending liquid, the only prerequisite is that the solid material does not dissolve in the liquid. If the liquid is volatile (Bp less than 100° C.), it can easily be dried in commercial dryers. If the liquid is less volatile, it can be washed with an appropriate volatile liquid during filtering. Total dryness in not needed if the carrier is used immediately in a wet-mixing process for manufacture the final formulation, as explained in the Finnish patent FI 105 078. It is possible to leave a certain fraction of polished particles in the final product. For example, a fraction of 20-30 μm polished particles can be returned to the main fraction. This can be done by selecting suitable filters to the filter pack so that the wanted fraction returns for retreatment. If no other means are used, the smallest particles, depending on the fine filter, will be present in the final product and are obviously located on the surface of the larger particles during the process or latest during drying. If the pore size of the finest filter is 2 microns, smaller carrier residues remain in the product and adhere on the surface of larger carrier particles during drying, preferably in a rotating evaporator. Also a soluble component may be added to the suspension for any reasons.

There are numerous ways to utilise polished carriers. As explained above, a combination of different fractions of polished carrier may be obtained for manufacture of the final formulation. The smaller size particles may act as ball bearings between the larger ones or they may form a separating layer on the surface of the larger particles. Then Van der Waals forces are diminished and disintegration of the drug particles is facilitated. The consequences can be easily studied by cascade impactor tests.

Polished particles may be coated with a secondary agent. Several methods are known including spray-drying with micronised or solvated secondary agent, gentle ball-milling with the agent (e.g. L-leucine and Mg-stearate) and gas-diffusion in vacuum. As reported, such coatings may greatly improve the flowability of carriers and also increase the FPF %. Polished carriers are excellent substrates for further developments.

The decisive idea in suspension-polishing is that vigorous modification of surfaces can be done without creating amorphous material. On the contrary, the surface layer with possible amorphous material is removed. The liquid acts as a coolant allowing only abrasion without melting or deforming the contact areas. The liquid also prevents agglomeration of particles of any size. If non-toxic liquids are used, toxicological hazards are avoided. A very important feature is that drastic enhancement in the properties can be done with physical treatment without using new chemical components, which should be proven safe for human inhalation before registration of the final medicinal product.

As the treatment can be carried out in a closed system using other than waterbased liquids, microbial contamination can be avoided.

The method is ready to be scaled-up in manufacturing scale. The manufacturer of the used dispersers informs that the system can be enlarged to any scale. The principle of the method is so simple that no risks in scale-up are seen. Also the cross-flow filter is in industrial use in filtering waste water.

1. Method for treating a particulate carrier for an inhalation powder improving stability and flow properties of the carrier, characterized in that carrier is abraded suspended in a liquid medium into which the carrier is essentially insoluble using an effect below that required for crushing the carrier particles, the liquid medium is removed and the carrier recovered.

2. Method according to claim 1, characterized in that the carrier is abraded with a mixing device.

3. Method according to claim 2, characterized in that the rotation speed of the mixing device is lowered during the treatment.

4. Method according to claim 3, characterized in that the carrier suspension is cooled and recirculated to the mixer.

5. A method according to claim 4, characterized in that the suspension is recirculated through a filter.

6. A method according to claim 5, characterized in that a certain desired size range or ranges are recirculated to the mixing device.

7. A method according to claim 1, characterized in that said media is a hydrocarbon, perfluorinated ether, fluorinated ether, perfluorinated hydrocarbon, fluorinated hydrocarbon, methanol, ethanol or any other alcohol or hydrocarbon.

8. A method according to claim 1, characterized in that said carrier after filtration is used undried for formulation.

9. A method according to claim 1, characterized in that said carrier is dried after filtration and stored for future use.

10. A method according to claim 1, characterized in that the abraded carrier is at least partly covered particles smaller in size than said carrier.

11. A method according to claim 10, characterized in that the abraded carrier and the small sized particles are of the same material.

12. A method according to claim 1, characterized in that the carrier to be abraded is lactose or a monohydrate thereof, glucose, mannitol, trehalose, sucrose, any other sugar, polysaccharide or any other compound used as a carrier.

13. Carrier for an inhalation powder, which carrier is stable and possesses good flowing properties, characterized in that the carrier is abraded suspended in a liquid medium, in which said carrier is essentially insoluble, and using an effect below that required for crushing the carrier particles.

14. Carrier according to claim 13, characterized in that that the carrier is abraded with a mixing device.

15. Carrier according to claim 13, characterized in that the carrier is filtrated and used for formulation undried or dried and stored for future use.

16. Carrier according to claim 15, characterised in that the filtrated carrier contains more than one main range of particle sizes of abraded carrier.

17. Carrier according to claim 1, characterized in that the carrier to be abraded is lactose or a monohydrate thereof, glucose, mannitol, trehalose, sucrose, any other sugar, polysaccharide or any other compound used as a carrier.

18. Preparation for inhalation purposes comprising an active agent, a carrier and optional excipients used in inhalable preparation, characterized in that at least a part of the carrier used is abraded suspended in a liquid medium, in which the carrier in essentially insoluble.

19. A preparation according to claim 18, characterized in that carrier contains more than one main range of particle sizes.
